(19) United States
(12) Patent Application Publication
(10) Pub. No.: US 2020/0359925 A1
(43) Pub. Date: Nov. 19, 2020

(54) ANALYSIS OF SPREADING DEPOLARIZATION WAVES

(71) Applicant: IMPERIAL COLLEGE OF SCIENCE, TECHNOLOGY AND MEDICINE, London (GB)

(72) Inventors: Martyn Gordon Boutelle, London (GB); Anthony J. Strong, London (GB); Sharon Jewel, London (GB)

(21) Appl. No.: 16/966,052

(22) PCT Filed: Feb. 5, 2019
(86) PCT No.: PCT/GB2019/050301

§ 371 Date: Jul. 30, 2020

(30) Foreign Application Priority Data
Feb. 5, 2018 (GB) 1801828.3

(51) Int. Cl. A61B 5/04; A61B 5/00; A61B 5/0478

(52) U.S. Cl. CPC A61B 5/04014; A61B 5/0478; A61B 5/7246; A61B 5/04004

(57) ABSTRACT

A method of automatically monitoring electrophysiological data in the brain and detecting clinically significant events comprises receiving signal inputs from at least one or more electrophysiological signal channels each indicative of electrical brain activity. For each of the one or more electrophysiological signal channels, the signals are filtered to obtain a first subchannel having a first frequency range and a second subchannel having a second frequency range. Appearance of a succession of correlated, non-synchronous events are detected in the waveforms of the one or more first subchannels to create a first detection output. Suppression of an amplitude of the signal is detected in one or more of the second subchannels correlated with the detected events in the one or more first subchannels to create a second detection output. The detected events are classified as a predetermined type of clinically significant event according to the first and second detection outputs. Spreading depolarization waves, peri-infarct depolarizations and other clinically significant events may be classified and displayed.

The invention relates to monitoring electrophysiological activity in the brain and, in particular though not exclusively, to analysis of brain activity to detect clinically significant events such as cortical spreading depression or spreading depolarization waves (hereinafter ‘SD waves’) and peri-infarct depolarizations (hereinafter ‘PIDs’).

The detection of certain types of clinically significant events within electrophysiological signals can be difficult to achieve for a number of reasons including: sensitivity to noise and interference in the signals; ambiguous signal features which may or may not be attributable to the specific clinically significant event types being monitored; and the complex nature of the signal features and their relationship with other corresponding signal features.

This can make accurate identification of certain types of clinically significant events very difficult even for experienced clinicians, who may be required to review many individual electrode traces, over extended periods of time, to identify patterns that may be indicative of the relevant clinically significant events. Significant features of the electrophysiological signals which could lead to the identification of a clinically significant event are not always easy to identify within the signal data.

It would be desirable to provide an automated system for analysing data sets received from brain electrodes, and optionally from other physiological sensors, and for providing accurate indications of clinically significant events derivable therefrom. It would also be desirable to provide an automated system which can monitor electrophysiological data signals in real time or pseudo-real time and provide continuing feedback on electrophysiological activity in the brain.

According to one aspect, the present invention provides a method of automatically monitoring electrophysiological data and detecting clinically significant events, comprising:

(i) receiving signal inputs from at least one or more electrophysiological signal channels each indicative of electrical brain activity;

(ii) for each of the one or more electrophysiological signal channels, filtering the signals to obtain a first subchannel having a first frequency range and a second subchannel having a second frequency range;

(iii) detecting the appearance of a succession of correlated, non-synchronous events in the waveforms of the one or more first subchannels to create a first detection output;

(iv) detecting the suppression of an amplitude of the signal in one or more of the second subchannels correlated with the detected events in the one or more first subchannels to create a second detection output;

(v) classifying the detected events as a predetermined type of clinically significant event according to the first and second detection outputs.

The first subchannels may have a frequency range substantially lower than the second subchannels. Step (i) may comprise receiving a plurality of said signal inputs from multiple said electrophysiological signal channels, each indicative of electrical brain activity. Step (iii) may comprise checking that each of the detected correlated, non-synchronous events in multiple ones of the first subchannels occurs within a specified time period of each other. Step (iii) may comprise creating a first detection output if the detected correlated, non-synchronous events in multiple ones of the first sub-channels occur in a series have an event rate within a predetermined range. The signal inputs may correspond to a plurality of electrocorticogram electrode signals from multiple adjacent electrodes sampled as a bipolar chain of adjacent pairs. In this case the correlated, non-synchronous events may comprise waveforms of alternating polarity in a sequence. Step (iv) may comprise detecting one of: (a) permanent suppression; and (b) temporary suppression. Step (v) may comprise classifying a detection output as a CSD event if the detected amplitude suppression in step (iv) is a temporary suppression. Step (v) may comprise classifying the detection output as a PID event if the detected amplitude suppression in step (iv) is a permanent suppression.

The method may further include: ascribing a confidence level for each first detection output in step (iii) and/or each second detection output in step (iv), and adjusting the confidence levels if a subsequent corresponding event is detected within a predetermined time window. The method may further include implementing step (v) only when one or more confidence levels has reached a predetermined threshold. Step (v) may comprise establishing a confidence level for each classified clinically significant event.

The method may further include, prior to the detecting steps, verifying the signals of the first and second subchannels are within the range of a data compliance test. The method may further include plotting, in real time, an event status for each of a succession of data epochs over time. Each event status may provide an indication of any detected events during the data epochs and a confidence level for each event status. The method may include retrospectively updating an event status for an earlier data epoch based on a status of a subsequent data epoch. The method may further include displaying detected events as a function of time correlated with other signals indicative of one or more of blood pressure, heart rate, mean arterial pressure, intracranial pressure, cerebral perfusion pressure, pressure reactivity, brain tissue oxygen, brain temperature, brain glucose, lactate/glucose ratio, brain potassium, brain sodium, pyruvate, patient motion. The signal inputs may comprise electrocorticography signals.

According to another aspect, the invention provides an apparatus for monitoring electrophysiological data and detecting clinically significant events, comprising:
an input module configured to receive signal inputs from at least one or more electrophysiological signal channels indicative of electrical brain activity;
a filter module configured to derive, for each of the one or more electrophysiological signal channels a first subchannel having a first frequency range and a second subchannel having a second frequency range;
a detection module configured to:
detect the appearance of a succession of correlated, non-synchronous events in the waveforms of the one or more first subchannels,
detect the suppression of an amplitude of the signal in one or more of the second subchannels correlated with the detected events in the one or more first subchannels;
a classification module configured to classify the detected events as a predetermined type of clinically significant event according to the output of the detection module.

According to another aspect, the invention provides an apparatus for monitoring electrophysiological data and detecting clinically significant events configured to carry out the various method steps as defined in the preceding paragraphs.

Embodiments of the present invention will now be described by way of example and with reference to the accompanying drawings in which:

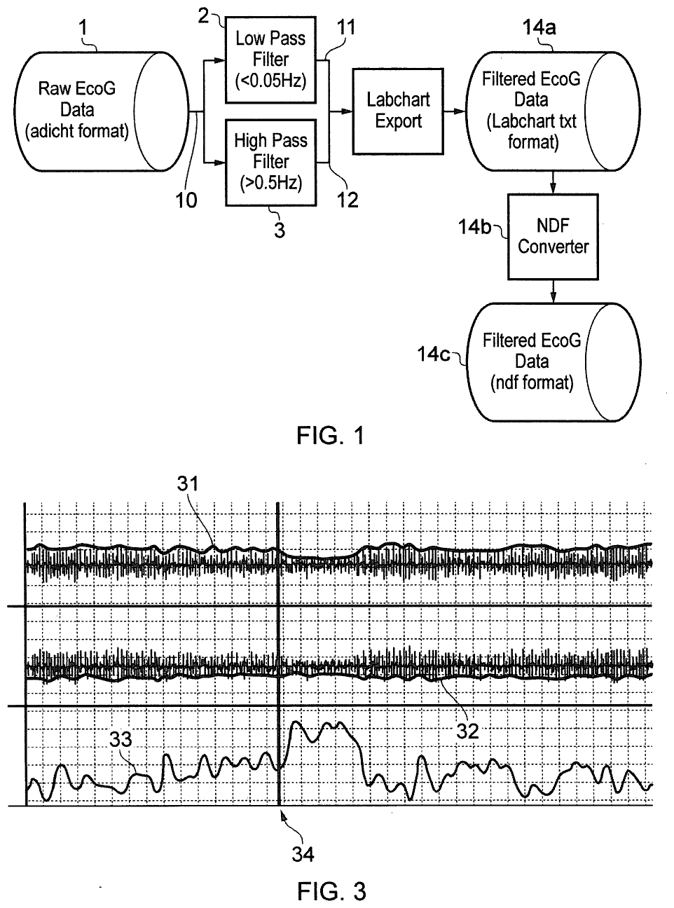

FIG. 1 shows a process flowchart of a method of pre-processing electrophysiological data for the detection of clinically significant events;

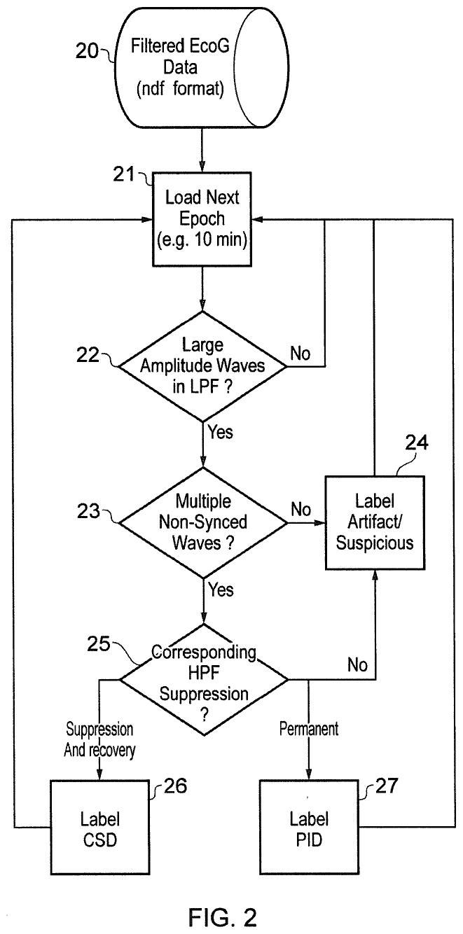

FIG. 2 shows a flowchart of a process for detecting and classifying clinically significant events;

FIG. 3 is a graph showing features of filtered electrophysiological data produced in the process of FIG. 2;

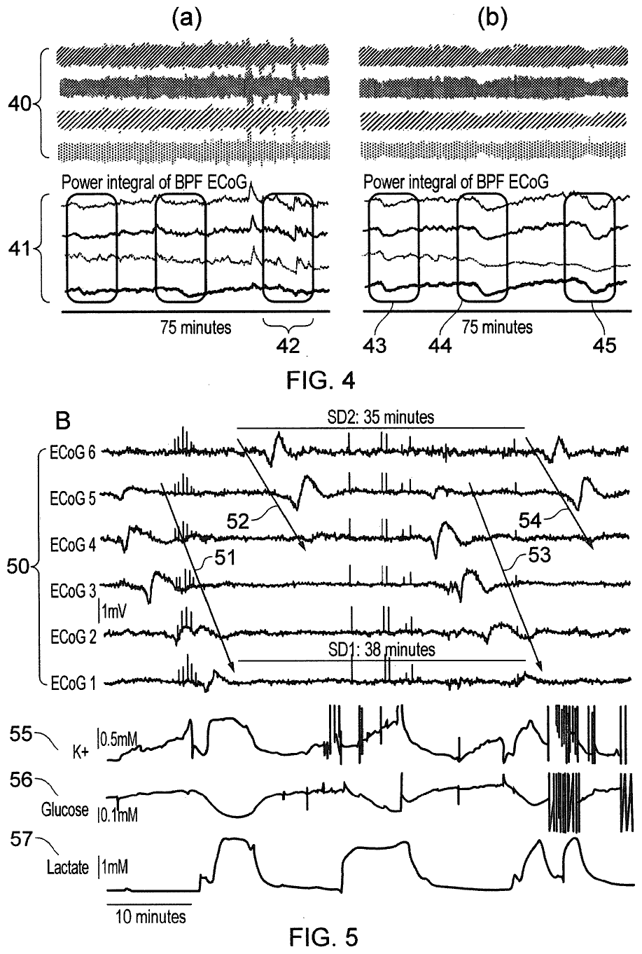

FIG. 4 shows electrophysiological data signals received from four electrodes inserted into the human cortex and corresponding power integral signals of higher frequency filtered subchannels therefrom;

FIG. 5 shows electrophysiological data signals from six ECoG electrodes illustrating spreading depolarization waves corresponding to clinically significant events and corresponding microdialysis data showing levels of potassium, glucose and lactate;

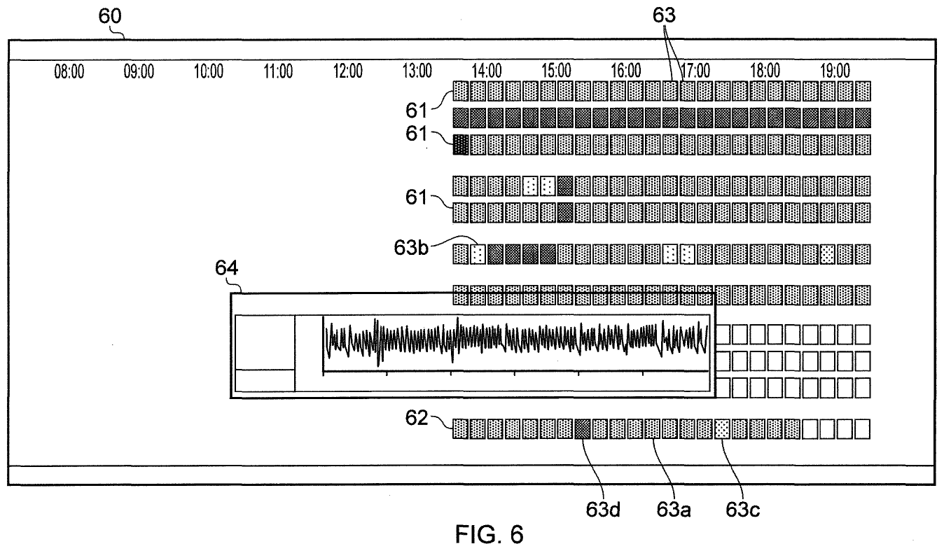

FIG. 6 shows an output of a neuromonitor display using data from the process of FIG. 2 together with other physiological sensed data.

An analysis tool as described herein is configured to detect clinically significant events in clinical datasets being received. Those clinical datasets may be received after a period of monitoring one or more individuals has been completed (e.g. in an ‘offline’ mode), or more preferably, may be processed ‘online’, e.g. continuously in real time or pseudo-real time (e.g. within a short period after the data has been sampled and on a continuing basis) and while the system is connected to monitoring electrodes.

Clinically significant events which may be detectable include spreading depolarization (SD) waves including cortical spreading depressions (CSDs) and peri-infarct depolarizations (PIDs). Other events which may be detected can include seizure activity and other changes coincident with or indicated by waves of spreading depolarization. The analysis tool may be configured to recognise and characterise SD waves and seizures in the electrophysiological data being received, and to characterise changes in neurochemical levels and brain pressure coincident with these waves.

The clinical datasets may comprise, or be derived from, electrocorticography (ECoG) electrodes placed directly on or into an exposed surface of the brain to record electrical activity from the cerebral cortex and may comprise strip electrodes extending over a surface of the brain tissue or depth electrodes inserted into the brain tissue. Electrode types may include electrode grids or custom electrode arrays having multiple electrode contacts at specific spacings. It may also be possible to use conventional electroencephalography electrodes monitoring electrical activity from outside the skull provided that sufficient signal can be obtained from which to extract the relevant data, to be discussed below.

For optimal detection of clinically significant events, preferably at least three, and preferably more, electrodes are used to provide at least three electrophysiological signal channels. In one arrangement, a bi-polar chain based on six electrodes is used, providing six electrophysiological signal channels corresponding to sampling from adjacent pairs of the electrodes. However, an SD wave may be detected in only a single channel using a single unipolar electrode with an appropriate reference, or two adjacent electrodes for a bipolar configuration.

The signal channels are first filtered using two different filters to obtain, for each of the electrophysiological signal channels, a first subchannel having a lower frequency range and a second subchannel having a higher frequency range.

The first subchannel filter may comprise a low frequency bandpass filter having a passband frequency range of 0.05 Hz to 30 Hz and the second subchannel filter may comprise a high frequency bandpass filter having a passband frequency range of 0.5 Hz to 30 Hz. The low frequency bandpass filter may have a lower cut-off frequency of 0.05 Hz, 0.02 Hz or even as low as 0.005 Hz. The lower cut-off frequency is in practice chosen to allow detection of the slow potential change while avoiding drift. SD events typically take 45 seconds seen in DC signals and 100 seconds seen as low frequency, near-DC signals, and as the passband lower cut-off frequency goes to lower frequencies the apparent magnitude of the slow potential change gets larger and changes shape, but eventually becomes difficult to detect due to false positives through baseline drift. In practice, the low frequency bandpass filter may have an upper cut-off frequency of up to the Nyquist frequency for the data sampling system, e.g. 200 Hz. However, more preferably is to bring the upper cut-off frequency down to less than mains power frequency, e.g. less than 50 or 60 Hz, e.g. 30 Hz or 45 Hz.

The second subchannel filter may comprise a bandpass filter having a bandpass frequency range of 0.5 to 30 Hz. In general, the high frequency bandpass filter preferably has a lower cut-off frequency of about 0.5 Hz such that it is high enough to discriminate against the slow waves captured by the low frequency bandpass filter, yet low enough to be sure to capture delta wave brain activity which may be typically 3 to 4 Hz, or possibly 0.5 to 4 Hz. In practice, the lower cut-off frequency could go as low as 0.2 Hz (i.e. a factor of ten greater than 0.02 Hz used in the lower frequency bandpass filter described above) or as high as 1.5 Hz. The upper cut-off frequency of the high frequency bandpass filter may be selected according to similar constraints relating to sampling rate and mains frequencies as defined above in relation to the low frequency bandpass filter, e.g. 30 Hz, 45 Hz, 200 Hz etc. The upper cut-off frequency of the low frequency bandpass filter could, of course, be set lower than the upper cut-off frequency of the high frequency bandpass filter, e.g. at 1 Hz or 0.5 Hz, to exclude the faster activity though in practice the magnitude of the signal at the higher frequency is sufficiently smaller than the slow wave signals as to be unlikely to adversely affect the measurements.

Thus, in the example using a six electrode array providing six channels, twelve subchannels may be obtained, comprising six low frequency subchannels (‘LPF’) and six high frequency subchannels (‘HPF’), e.g.

1. LPF1 using electrode pair 1 and 2

2. LPF2 using electrode pair 2 and 3

3. LPF3 using electrode pair 3 and 4

4. LPF4 using electrode pair 4 and 5

5. LPF5 using electrode pair 5 and 6

6. LPF6 using electrode pair 6 and 1

7. HPF1 using electrode pair 1 and 2

8. HPF2 using electrode pair 2 and 3

9. HPF3 using electrode pair 3 and 4

10. HPF4 using electrode pair 4 and 5

11. HPF5 using electrode pair 5 and 6

12. HPF6 using electrode pair 6 and 1

For a strip electrode, the electrode pair 6 and 1 may be less useful if they are located at opposite ends of the strip.

Other numbers of electrodes and electrode configurations can be used. For example, individual electrodes could be referenced against a common electrode rather than to an adjacent electrode. The common electrode could be one or more of the strip, grid or probe electrodes (e.g. on the same substrate or an internal reference electrode) or could be a remotely located reference electrode (e.g. a far field common reference). The common electrode could be implemented using mean referencing or n−1 referencing, where the electrode being sampled is compared with the mean signal from all (or some) of the other electrodes/channels as a reference. Noise which appears on all channels will thus be subtracted while a signal which appears only on the subject channel will appear as output. This technique is particularly useful with depth electrodes. For depth electrodes, it may be optimal to choose the deepest electrode as an internal reference as it may be the most silent.

Thus, in a general aspect, the arrangements above exemplify receiving signal inputs from one or more electrophysiological signal channels, each channel indicative of electrical brain activity and, for each of the one or more electrophysiological signal channels, filtering the signals to obtain a first subchannel having a first frequency range (e.g. a lower frequency range) and a second subchannel having a second frequency range (e.g. a higher frequency range).

FIG. 1 illustrates a schematic example of the above process, in which raw data signals 1 are received to provide electrophysiological data signal channels 10, which are fed to a low pass filter 2 and a high pass filter 3 to generate first subchannels 11 and second subchannels 12. In the off-line analysis environment depicted in FIG. 1, the data from these subchannels is exported and format-converted from, e.g. a Labchart format to a Neurophysiology Data Format (NDF) as shown at 14a, 14b, 14c. Any suitable data signal format processing as may be required can be envisaged, whether for offline or online processing, ensuring that signal data is adequately time-stamped.

FIG. 2 illustrates an example of a detection process carried out on the subchannel data 11, 12. This exemplary process is configured to detect and differentiate between cortical spreading depressions (CSDs) and peri-infarct depolarizations (PIDs).

The subchannel datasets are received at step 20. Prior to commencement of the process as shown in FIG. 2, a data compliance test (not shown) may be applied to the subchannel datasets to ensure that the data in any given subchannel is reasonable. The data compliance test may verify that the filtered signals are within the range of physiologically plausible values. The compliance ranges can be configured to exclude sudden signal excursions caused by, for example, noise, interference, electrode displacement or disconnection, body movement etc. The compliance test may verify whether changes within a subchannel are slow enough to be realistically biological. Any subchannels which are determined to contain bad data may be excluded from subsequent processing, e.g. at least temporarily or until a signal stabilizes to compliant values. This data compliance test may avoid the detection of many undesirable artefacts. A data compliance test may be supported by reference to an accelerometer giving indications of sudden movement of the monitored patient which could be used to temporarily blank data signals.

Detection of a clinically significant event relies on multiple signal events occurring in the subchannels' signals within a short time frame or specified time period. Parameters that may be considered may include (i) a time period between correlated events within a channel, (ii) a time period between correlated events across different channels (e.g. adjacent channels). The precise timing of the events in at least scenario (ii) above may be influenced by electrode configuration, e.g. electrode separation.

By way of example, for a strip electrode with a centre-to-centre contact space of 1 cm, the time period in (ii) is 10 seconds or less and for a depth electrode, the time period in (ii) is less than 5 seconds. For events travelling across multiple channels, a wave speed is typically 2 to 3 mm per minute (minimum 0.5 mm/min, maximum 7 mm/min). If the contact spacing were to be 1 cm centre-to-centre (for 2.4 mm radius contact) then the wave could take up to 10 minutes at 0.5 mm/min to cover the 5 mm gap. Some variability may be allowed for as there may be, for example, a sulcus in between contacts, or there could be more than one wave detected on the electrode strip.

One approach as described here is based upon a sliding window. The contents of each window (data epoch, step 21) are examined across the various filtered subchannels, looking for (amongst other things) high amplitude, low frequency waves (step 22) and high frequency suppression (step 25). A next data epoch is then loaded (step 21), as the window slides across the dataset. When implementing a real-time system, this approach is still realistic, with detections being based on current events and a set period of time in the past. In one example, this period (and hence the size of the sliding window) may be of the order of 10 minutes, based upon the required proximity of low level events to suggest that a CSD or PID has occurred.

The detection of CSD and PID events may depend on two lower level types of event: slow potential changes (SPCs—low frequency, large amplitude waves) and high frequency amplitude suppression. The first event required is a slow potential change in the low pass filtered subchannel data (step 22) as discussed below.

If no such waves are present in a data epoch, the possibility of CSD and PID events can be safely discounted and the process returns to step 21. If an SPC is present, further investigation is required, as shown in FIG. 2. The system tests for multiple, non-synchronous events in the low frequency subchannels 11. If only a single wave is present in an epoch, this is not sufficient for detection of any CSD or PID events. The presence of such a wave can still be noted as ‘suspicious’ (step 24), but it is likely an artefact. If multiple waves are present (step 23), their synchronicity is examined. If the waves are highly synchronised (e.g. substantially aligned in time) then the event should also be noted only as ‘suspicious’ (step 24), and not as anything more. This is because a clinically significant biological event is highly unlikely to be extremely synchronous due to the slow rate at which the waves travel.

Slow potential changes manifest as waves which have a high amplitude compared to the local background. The amplitude of these waves can be expected to be in the range 0.4-4 mV peak to peak, with over 1 mV being usual. These waves should be detectable using a combination of filters which emphasise the waves compared to the background.

One criterion is that the onset of a slow potential change should be very slow, e.g. of the order of one minute in duration. Any case in which the onset occurs more rapidly may be dismissed as an artefact. Repeated slow potential changes for a single patient tend to show significant stereotyping, where the shape of a slow wave (slow potential change) looks the same when it repeats on the same channel. It might also look the same on an adjacent channel, but this is not common. Therefore, it may be desirable to use repeated detections to increase confidence in past or future detections based upon the shape and timing of the wave.

In the case of signals derived from bipolar configuration electrode data such as described above, each slow potential change should appear on two adjacent channels, inverted on one. This is because the data is recorded in a bipolar chain, with each electrode appearing in two channels. If multiple asynchronous waves are present, this is classified as highly suspicious of either a CSD or PID event. Examination of the high pass filter data will determine how the event should be classified.

In a general aspect, steps 20 to 23 of FIG. 2 exemplify a process of detecting the appearance of a succession of correlated, non-synchronous events in the waveforms of one or more, and preferably multiple ones, of the first subchannels to create a first detection output (e.g. the positive output from step 23).

The higher frequency subchannels 12 respectively corresponding to the subchannels 11 on which slow potential changes were detected are examined (step 25). Three possible features in the higher frequency subchannel signals may be expected: (i) suppression of the high frequency amplitude, followed by recovery; (ii) permanent suppression of the high frequency amplitude with little or no signal evident; and (iii) no suppression of the high frequency amplitude.

If feature (i) is observed on one or more subchannels 12, the event should be classified as a CSD (step 26). If feature (ii) is observed, the event should be classified as a PID (step 27). If feature (iii) is observed, the event should be labelled only as ‘suspicious’ (step 24).

In the case that some subchannels 12 contain suppression and recovery (feature (i)) and other subchannels show permanent suppression (feature (ii)), the event should be labelled as a PID (step 27). The two forms of high frequency depression that may be detected may be described as permanent suppression, and temporary suppression, i.e. suppression and recovery. The expression ‘temporary’ may be defined as encompassing the scenario where the suppression starts to recover before a next SD wave arrives (typically 20 to 30 minutes), and recovery can typically occur within 5 or 10 minutes. The expression ‘permanent’ may be defined as encompassing the scenario where there is no sign of recovery before the next wave arrives, if there is one, e.g. within or longer than 20 to 30 minutes. Thus, the expression ‘permanent’ may defined as greater than 30-40 minutes where no further SD wave arrives. The system of course would not detect any suppression events if there were no slow potential change to trigger the test. The system learns from what is happening and changes its confidence about these and also its categorisation of the events.

Suppression and recovery may be detected through the use of a pair of envelope filters and a difference filter. One envelope filter is set to trace the top of the HPF data (trace 31), and one the bottom of the HPF data (trace 32), as shown in the top two channels in FIG. 3.

The difference between these two envelopes is then calculated (trace 33), as shown in the bottom channel of FIG. 3. This highlights the positions in the data where the suppression occurs, as can be clearly seen at the temporal position marked with the vertical line at 34.

In a general aspect, the process of steps 25 to 27 exemplifies detecting the suppression of an amplitude of the signal in one or more of the second subchannels correlated with the detected events in the at least one or more first subchannels to create a second detection output (at step 25), and classifying the detected events as a predetermined type of clinically significant event according to the first and second detection outputs.

The location of detected clinically significant events may be marked for the subchannels where they occur and in other subchannels in the data set. A confidence level may be determined for each event. A summary of the events found may be provided including, for example: a type of event, a start and end time, a level of confidence in the classification and, where appropriate, a duration of the suppression in each of the higher frequency subchannels.

A confidence level may be ascribed for each first detection output and/or for each second detection output and/or for each classified clinically significant event. Confidence levels may be adjusted if a subsequent corresponding event is later detected within a predetermined time window. In an example, a confidence level for a classified clinically significant event may be established only when one or more of the confidence levels of the first detection output and/or the second detection output reach a corresponding predetermined threshold.

FIG. 4, top four traces 40, illustrate electrophysiological data signals in subchannels 12 corresponding to the higher frequency data received from four electrodes of a depth electrode array inserted into the human cortex. In this example, the signals extend over a time interval of 75 minutes. The lower four traces 41 show a power integral of the data. The left hand data set of FIG. 4a illustrates data recorded with a remote reference and the right hand data set of FIG. 4b illustrates the data processed to use mean referencing. The substantially noisy signal during the period indicated at 42 of FIG. 4a has been cleaned in FIG. 4b leaving a clear indication of the amplitude suppression visible in each of the rectangular box overlays 43, 44, 45.

FIG. 5, top six traces 50, show ECoG data signals from subchannels 11 (the lower frequency subchannels) illustrating repetitive spreading depolarizations collected from a traumatic brain injury patient. The signals show a total of four SD waves 51, 52, 53, 54 indicated by arrows 51-54. The top six traces 50 show the large slow potential change in the low frequency (near-DC) current ECoG data. As seen in the figure, each of these four SD waves 51-54 exemplifies a succession or series of correlated, non-synchronous events in multiple ones of the first subchannels.

The correlation of the events may be determined according to a number of properties, including: a similar shape of wave profile appearing in two or more channels (e.g. all matching a predetermined template or all matching a set of wave profile parameters); a small time separation between each wave profile appearing in each adjacent or near adjacent channel signal; a repeating cycle in subsequent data epochs. In the example of FIG. 5, the repeating cycles have a cycle length of 35 and 38 minutes respectively. As more generally discussed above, repeating cycles within a channel may have a cycle time of 15 minutes or considerably longer, for a series of SD waves passing the contacts in the same direction. If the wave reverses, it may have a different shape and may be detected with less confidence, but if it repeats in the new direction, the stereotyping may increase and confidence levels will built again.

The series of correlated non-synchronous events in different channels may be checked to see if they comply with an ‘event rate’ (e.g. a number of events per unit time—which is approximated by the angle of the arrows 51-54) that lies within a predetermined range of allowable event rates.

The bottom three traces 55, 56, 57 show a corresponding tissue response from microdialysate data: potassium (trace 55), glucose (56) and lactate (57) which corroborate the determination of a clinically significant event from the electrophysiological data sets.

Detected clinically significant events may be displayed, e.g. in real time, on a suitable display device. An example is shown in FIG. 6.

The display 60 of FIG. 6 comprises a plurality of rows 61, each corresponding to detected events from a plurality of monitored physiological parameters. These physiological parameters include clinically significant events in electrophysiological data sets as detected by the analysis tool described above, e.g. in row 62. Each row 61, 62 may be divided into time blocks 63 of, for example, 15 minutes. Each block 63 may have a status indicated by its colour. An absence of detected SD events may be represented by a green colour, e.g. block 63a. Events that have been categorised as ‘suspicious’ but not determined to be clinically significant events may be represented by a different colour, e.g. yellow block 63b. When further events are detected later in the signal, e.g. in a later data epoch, which tend to confirm a suspicious event as a likely clinically significant event, the status can be updated retrospectively to a further colour or colours, e.g. orange (block 63c) or red (block 63d) depending on the confidence level of the categorization of the event. The display 60 may be configured to extend over any suitable period of time, e.g. 12 hours or one or more working shift periods in a clinical environment. The display 60 may be configured to display data on a rolling basis, e.g. the previous 12 hours.

The display 60 may be configured to display detected events of one or more other physiological parameters derived from other sensors monitoring a patient. Examples of other parameters may include one or more of blood pressure, heart rate, mean arterial pressure, intracranial pressure, cerebral perfusion pressure, pressure reactivity, brain tissue oxygen, brain temperature, brain glucose, lactate/glucose ratio, brain potassium, brain sodium, pyruvate, patient motion (e.g. sensed by a three-axis accelerometer). Detected events in the other physiological signals may be triggered by levels that have been previously established to be adverse to a patient.

Detected events in the other physiological parameters may also be used to modify confidence levels in detected events in the electrophysiological event data in row 62.

As shown in FIG. 6, the display 60 may be configured such that selection of a block 63 using a convention graphical user interface (e.g. mouse or touchscreen) may open a window 64 showing underlying raw data or part-processed data, to enable a clinician to examine the underlying data that led to classification of clinically significant events.

Other embodiments are intentionally within the scope of the accompanying claims.

1. A method of automatically monitoring electrophysiological data and detecting clinically significant events, comprising:
(i) receiving signal inputs from at least one or more electrophysiological signal channels each indicative of electrical brain activity;
(ii) for each of the one or more electrophysiological signal channels, filtering the signals to obtain a first subchannel having a first frequency range and a second subchannel having a second frequency range;
(iii) detecting the appearance of a succession of correlated, non-synchronous events in the waveforms of the one or more first subchannels to create a first detection output;
(iv) detecting the suppression of an amplitude of the signal in one or more of the second subchannels correlated with the detected events in the one or more first subchannels to create a second detection output;
(v) classifying the detected events as a predetermined type of clinically significant event according to the first and second detection outputs.

2. The method of claim 1 in which the first subchannels have a frequency range substantially lower than the second subchannels.

3. The method of claim 1 in which step (i) comprises receiving a plurality of said signal inputs from multiple said electrophysiological signal channels, each indicative of electrical brain activity.

4. The method of claim 3 in which step (iii) comprises checking that each of the detected correlated, non-synchronous events in multiple ones of the first subchannels occurs within a specified time period of each other.

5. The method of claim 4 in which step (iii) comprises creating a first detection output if the detected correlated, non-synchronous events in multiple ones of the first sub-channels occur in a series have an event rate within a predetermined range.

6. The method of claim 3 in which the signal inputs correspond to a plurality of electrocorticogram electrode signals from multiple adjacent electrodes sampled as a bipolar chain of adjacent pairs and in which the correlated, non-synchronous events comprise waveforms of alternating polarity in a sequence.

7. The method of claim 1 in which step (iv) comprises detecting one of: (a) permanent suppression; and (b) temporary suppression.

8. The method of claim 7 in which step (v) comprises classifying a detection output as a CSD event if the detected amplitude suppression in step (iv) is a temporary suppression, and classifying the detection output as a PID event if the detected amplitude suppression in step (iv) is a permanent suppression.

9. The method of claim 1 further including:
ascribing a confidence level for each first detection output in step (iii) and/or each second detection output in step (iv), and
adjusting the confidence levels if a subsequent corresponding event is detected within a predetermined time window.

10. The method of claim 9 further including implementing step (v) only when one or more confidence levels has reached a predetermined threshold.

11. The method of claim 1 in which step (v) comprises establishing a confidence level for each classified clinically significant event.

12. The method of claim 1 further including, prior to the detecting steps, verifying the signals of the first and second subchannels are within the range of a data compliance test.

13. The method of claim 11 further including plotting, in real time, an event status for each of a succession of data epochs over time, each event status providing an indication of any detected events during the data epochs and a confidence level for each event status.

14. The method of claim 13 further including retrospectively updating an event status for an earlier data epoch based on a status of a subsequent data epoch.

15. The method of claim 1 further including:
displaying detected events as a function of time correlated with other signals indicative of one or more of blood pressure, heart rate, mean arterial pressure, intracranial pressure, cerebral perfusion pressure, pressure reactivity, brain tissue oxygen, brain temperature, brain glucose, lactate/glucose ratio, brain potassium, brain sodium, pyruvate, patient motion.

16. The method of claim 1 in which the signal inputs comprise electrocorticography signals.

17. Apparatus for monitoring electrophysiological data and detecting clinically significant events, comprising:
an input module configured to receive signal inputs from at least one or more electrophysiological signal channels indicative of electrical brain activity;
a filter module configured to derive, for each of the one or more electrophysiological signal channels a first subchannel having a first frequency range and a second subchannel having a second frequency range;
a detection module configured to:
detect the appearance of a succession of correlated, non-synchronous events in the waveforms of the one or more first subchannels,
detect the suppression of an amplitude of the signal in one or more of the second subchannels correlated with the detected events in the one or more first subchannels;
a classification module configured to classify the detected events as a predetermined type of clinically significant event according to the output of the detection module.

18. Apparatus for monitoring electrophysiological data and detecting clinically significant events configured to carry out the steps of claim 1.
